(19) United States
(12) Patent Application Publication
(10) Pub. No.: US 2006/0089538 A1
(43) Pub. Date: Apr. 27, 2006

(54) Device, system and method for detection activity of persons

(75) Inventors: Paul Edward Cuddihy, Ballston Lake, NY (US); Jenny Marie Weisenberg, Niskayuna, NY (US); Robert Nelson Ludlow, Oconomowoc, WI (US); Steven Phillip Roehm, Waukesha, WI (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/969,864

(22) Filed: Oct. 22, 2004

(51) Int. Cl. A61B 5/00; A61B 5/103

(52) U.S. Cl. 600/300; 600/595; 128/903

(57) ABSTRACT

A device, system and method for monitoring the activity of one or more residents in a habitable structure so as to ascertain whether the activity, or inactivity, is unusual. A variety of sensors distributed around the home collect data on activity in the home. The monitored person or persons wear an independent activity detector that monitors individual activity of a specific person. The house data is communicated to a remote monitoring center, and the individual data is communicated to an analyzing mechanism, which may be a communication panel or the remote monitoring center. The house data and individual data are analyzed and compared to ascertain whether any specific person within the habitable structure is engaged in unusual activity or inactivity. The independent activity detector may be configured to transmit an authentication signal to authenticate specific detected activity with a specific person.

### BACKGROUND

The invention relates generally to a device, system and method for monitoring the in-home activities of persons living independently. More particularly, the invention relates to a device, system and method for determining whether the activity or inactivity of one or more persons residing within a habitable structure is unusual.

With medical advancements and increased attention to proper nutrition and sufficient exercise, the populace in the western civilization is living longer. For example, the number of elderly persons residing in the United States is increasing, and with the advancing age of the baby boomer generation, the number of elderly persons in the United States will increase significantly over the next several decades. Additionally, increased awareness and understanding of various mental and physical disabilities has led to an increase in the number of persons living independently that are known to have diminished mental and/or physical faculties.

With the increase in elderly and disabled persons living independently has come anxiety that these elderly and disabled persons are safe and secure in their own residences. There is increased anxiety by the elderly and disabled living alone that they may become injured or incapacitated and be unable to summon assistance. That anxiety is often shared by loved ones living at a distance from the elderly and/or disabled living independently.

Currently, the anxiety felt by the elderly and disabled living alone, as well as the anxiety felt by their loved ones, is addressed through several avenues. One way to ease anxiety is through frequent visits to the home by a caregiver. Such visits can be intrusive, time consuming, and often inconvenient and not appreciated. Another way is for the elderly or disabled person to move out of the home and move into a facility better able to monitor his health. This, however, strips the person of his independence, is costly and is often unwelcome. Another way is through technological assistance or monitoring of the person in the home.

Such technological systems that assist persons in their home include Personal Emergency Response Systems. In these systems the elderly or disabled individual wears a watch, pendant or other like device and presses a button in the event of an emergency, such as a fall. The depressed button enables an alarm signal. A central monitoring facility provides assistance by responding to the alarm signal and calls the individual to identify the problem. The facility can also call a predetermined list of contacts, such as relatives, neighbors or emergency services, as required by the context of the situation. While a valuable service, these systems only identify problems that occur when the individual is able to press the emergency button.

Some known in-home monitoring systems attempt to detect unusual activity, such as, for example, an abnormally long quiet period within the house, by arbitrarily choosing “daytime” and “nighttime” parameters and sending an alert if there is no activity for at least five hours during the “daytime” time period. Such known systems oftentimes fail to accurately reflect real daytime and nighttime patterns of the resident.

Another known technological system includes a device worn by the monitored person. The device collects short-term data over an extended period of time, such as for a period of forty-five days. The data is then retrieved from the device to diagnose the activity detected. A disadvantage of such a system is that there is no remote connectivity to the device and the retrieval of the data is not performed near enough to real time. Another known technological system includes a device to be worn by the monitored person. The device includes processing capability to enable it to perform processing of the data. The device then transmits the processed data out. Disadvantages to such a system are the high cost and the loss of the raw data through the local processing of the data. Another technological system includes only using motion sensors, which provides lower resolution and fails to distinguish between individual persons within a home.

Thus, there remains a need for a system and method for ascertaining whether the in-home activities of a person living independently are within the norm for such a person or whether they are unusual or irregular.

### BRIEF DESCRIPTION OF THE DRAWINGS

FIG. 1 is a schematic view of an independent activity detector system in accordance with an exemplary embodiment of the invention.

FIG. 2 is another schematic representation of the system of FIG. 1.

FIG. 3 is a graph illustrating the overlapping of information in data packets transmitted from the independent activity detector of FIG. 1.

FIG. 4 illustrates process steps for determining with the independent activity detector of FIG. 1 whether activity of a person is unusual.

FIG. 5 illustrates an in-home monitoring system constructed in accordance with another exemplary embodiment of the invention.

FIG. 6 illustrates activity data obtained through motion sensors in a home.

FIG. 7 illustrates activity data obtained through the independent activity detector of FIG. 1.

FIG. 8 illustrates process steps for determining with the in-home monitoring system of FIG. 5 whether activity of a person is unusual.

### SUMMARY

The invention is directed to a system and a method for determining the activity or inactivity of one or more persons residing within a habitable structure, such as a house.

One exemplary embodiment of the invention provides an independent activity detector configured to be associated with a person. The independent activity detector includes an activity detection module configured to collect data on activity of the person, a storage mechanism configured to store the data collected by the activity detection module, and a transmitter configured to transmit in at least quasi-real time the data collected by the activity detection module. The storage mechanism is configured to retain a packet of data transmitted by the transmitter for retransmission by the transmitter.

An aspect of the independent activity detector embodiment provides for an independent activity detector to include an activity detection module, a storage mechanism, and a transmitter. The transmitter is configured to transmit unfiltered, unanalyzed and in quasi-real time the data collected by the activity detection module. The storage mechanism is configured to retain a packet of data transmitted by the transmitter for retransmission by the transmitter.

Another exemplary embodiment of the invention provides an independent activity detector system for use within a habitable structure. The independent activity detector system includes an independent activity detector configured to be associated with a person and a communication relay. The independent activity detector includes an activity detection module configured to collect data on activity of the person, a storage mechanism configured to store the data collected by the activity detection module, and a transmitter configured to transmit in at least quasi-real time the data collected by the activity detection module. The storage mechanism is configured to retain a packet of data transmitted by the transmitter for retransmission by the transmitter. The communication relay is configured to receive the data transmitted by the transmitter.

An aspect of the independent activity detector system embodiment provides for an independent activity detector system that includes an independent activity detector configured to be associated with a person and a communication relay. The independent activity detector includes an activity detection module configured to collect data on activity of the person and configured to ascertain whether the person is motionless, a storage mechanism configured to store the data collected by the activity detection module, and a transmitter configured to transmit unfiltered, unanalyzed and in quasi-real time the data collected by the activity detection module. The storage mechanism is configured to retain a packet of data transmitted by the transmitter for retransmission by the transmitter.

Another exemplary embodiment of the invention is an independent activity detector system for monitoring a person's health. The independent activity detector system includes an independent activity detector configured to be associated with a person and a physiological sensor for obtaining physiological data pertaining to the person. The independent activity detector includes an activity detection module configured to collect activity data on activity of the person, a storage mechanism configured to store the activity data collected by the activity detection module, and a first transmitter configured to transmit unfiltered, unanalyzed and in at least quasi-real time the activity data collected by the activity detection module. The independent activity detector system also includes a second transmitter configured to transmit physiological data collected by the activity detection module and a communication relay configured to receive the activity and physiological data transmitted by the first and second transmitters. The physiological data and the activity data are correlated to determine a status of at least one physiological condition of the person.

Another exemplary embodiment of the invention is a method for detecting unusual activity or inactivity of a person within a habitable structure. The method includes detecting individual activity data of the person with an independent activity detector, transmitting the individual activity data in at least quasi-real time to an analyzing mechanism, and ascertaining whether activity or inactivity of the person detected with the independent activity detector is unusual.

Another exemplary embodiment of the invention is a method for detecting unusual activity or inactivity of a person within a habitable structure. The method includes detecting activity data in the habitable structure, detecting individual activity data of the person, transmitting the individual activity data in quasi-real time to an analyzing mechanism, comparing the activity data of the habitable structure to the individual activity data, and checking for unusual activity or unusual inactivity of the person.

These and other advantages and features will be more readily understood from the following detailed description of preferred embodiments of the invention that is provided in connection with the accompanying drawings.

### DETAILED DESCRIPTION OF EXEMPLARY EMBODIMENTS

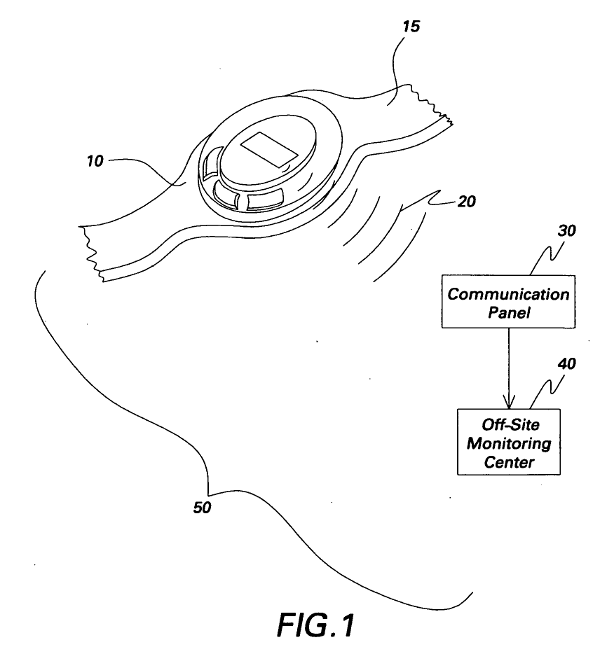

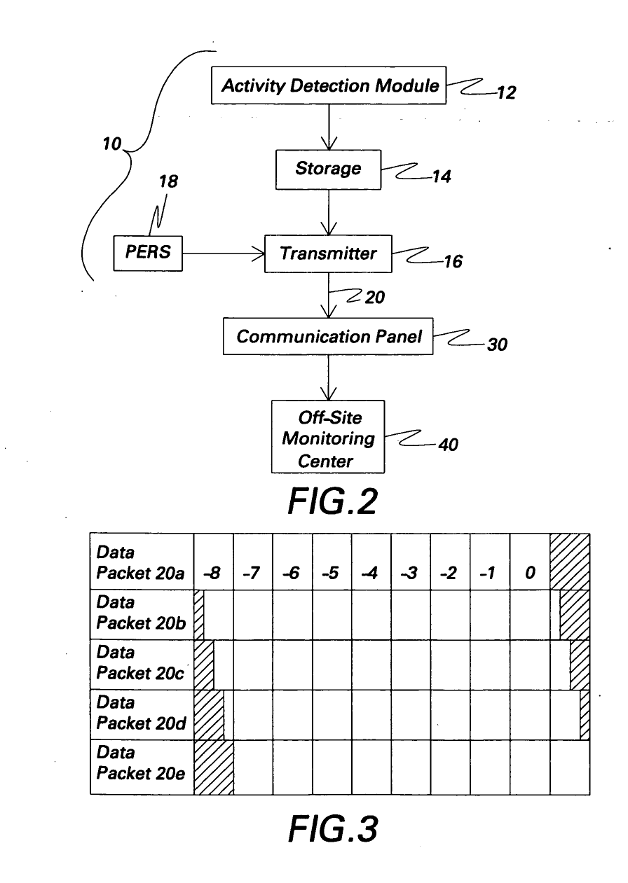

With reference to FIGS. 1-2, there is shown an independent activity detector system 50 that includes an independent activity detector 10, a communication panel 30 and an off-site monitoring center 40. The independent activity detector 10, configured to be worn by a person for whom activity data is to be determined, is enabled to measure one or more of movement, acceleration, vibration, and change in orientation. The independent activity detector 10 may be equipped with a chain or other such apparatus for hanging the detector 10 from one's neck. Alternatively, and as illustrated, the independent activity detector 10 includes a strap 15 for attaching to a person's wrist. The independent activity detector 10 may be incorporated within a timepiece, such as a watch. It should be appreciated that the independent activity detector 10 may be equipped with other means of wearing the device instead of the strap 15, such as, for example, a hook, clip, pin, etc. so that the device may be worn on a person's belt or clothing. It should be appreciated that any mechanism for associating the independent activity detector 10 to one particular person is within the scope of the invention, including adhesives, mechanical attachment devices, embedding within the person's clothing, or other suitable mechanism.

The independent activity detector 10 includes an activity detection module 12, a storage 14, and a transmitter 16. The activity detection module 12, which is configured to detect the activity of the person wearing the independent activity detector 10, may be any suitable such apparatus, such as, for example, a mercury switch or an accelerometer. It should be appreciated that this list of possible activity detection modules 12 is not exhaustive, and is merely representative of the type of devices that may be used. The storage 14 is configured to store all the data collected by the activity detection module 12 until such time that the data is transmitted from the independent activity detector 10. Further, the storage 14 is configured to retain packets of data for a period of time after transmission of such data. The storage 14 may be any suitable memory apparatus, such as, for example, a RAM memory chip.

The transmitter 16 is configured to wirelessly transmit data from the independent activity detector 10 to a receiver. Prior to transmission, no analysis of the data is performed. By analysis is meant that no determination is made to make qualitative judgments regarding the data, i.e., whether any particular data is particularly significant or insignificant. Further, the data is transmitted without any filtering of the data. The detector 10 may also include a receiver (not shown) for receiving an acknowledgement of its transmission. Having such a receiver on the detector 10 enables the detector 10 to transmit only the necessary data.

The receiver for the transmission from the transmitter 16 is a communication relay, such as the communication panel 30. It should be appreciated that the communication relay may be a wireless hub. With specific reference to FIG. 3, next will be described the transmission of data from the independent activity detector 10 to the communication panel 30. Transmission of data is done through a series of data packets 20 sent in the transmission. Each transmission includes an authentication code or signal along with the data packet 20 that identifies the specific independent activity detector 10, which is itself correlated with a particular person. The data packets 20 are transmitted at intervals from each other, for example, at intervals of fifteen minutes. For example, at time 0 a first data packet 20a is transmitted in a transmission. The data packet 20a may include data from, for example, the previous eight hours (from time −8 to 0). Approximately fifteen minutes later, a second data packet 20b is transmitted. The oldest fifteen minutes of data transmitted in data packet 20a is not included with data packet 20b, but a new fifteen minutes not included in data packet 20a is included with data packet 20b. Hence, there is an overlap of seven and three quarter hours of data between data packet 20a and data packet 20b, i.e., seven and three quarter hours of data is stored in storage 14 and retransmitted. Data packets 20c, 20d and 20e are sequentially transmitted every fifteen minutes, or at whatever interval is chosen. Data packet 20e has an overlap of data with data packet 20a of seven hours, and includes one new hour of data not included in data packet 20a. Providing a memory for eight or more hours of data enables a person to leave the home, and upon return, at least a portion of the data of activities during that time period outside the home can be retrieved. While a storage period of eight hours has been described as exemplary, it should be appreciated that the length of the period is not restricted to any particular length.

A personal emergency response system (PERS) 18 may be included in the independent activity detector 10. A person who has fallen and may be injured but is still conscious and capable of activating the PERS 18 may activate the PERS 18. Regardless of whether the PERS 18 is activated upon a fall, or upon any unusual activity or inactivity, the independent activity detector system 50 can detect unusual activity or inactivity on a quasi-real time basis.

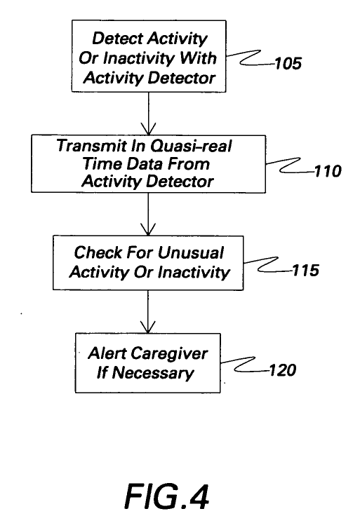

Referring specifically to FIGS. 1, 2 and 4, next will be described a method for detecting activity utilizing the independent activity detector system 50. At Step 105, the independent activity detector 10 detects activity or inactivity of the individual wearing the device. Utilizing an activity detection module 12, such as an accelerometer, a mercury switch, or other suitable activity-detecting apparatus, the independent activity detector 10 collects data pertaining to the activity or inactivity and stores such data in a storage module 14.

At Step 110, the collected data is wirelessly transmitted in an unfiltered format from the storage module 14 to a communication relay (panel 30) via a transmitter 16. Each transmission includes an authentication signal or code as well as a data packet 20. The transmissions are accomplished in quasi-real time, namely within a short period of time of the collection of the data. The period of time between collection and transmission of data may be chosen, but should be no more than about one hundred and twenty (120) minutes, and preferably should be fifteen or less minutes. It should be appreciated that the communication panel 30 may improve the efficiency of the transmissions without making any sort of judgment as to the contents of any particular data packet 20. For example, the communication panel 30 may buffer transmitted data over a time period, such as fifteen minutes. It also should be appreciated that certain factors impinge upon the ability to collect and transmit data, including but not limited to, battery life and the transmitting frequencies. Provided battery life can be enhanced, and provided that one or more transmitting frequencies are selected allowing for more continuous radio transmission of data, the time period for quasi-real time transmission may be lessened and may approach a real time transmission. Thus, with sufficient power, it may be possible to collect and transmit the data on a real-time basis. A shorter period of time between the collection and the transmission of data enables one monitoring the data to better and more quickly ascertain irregularities in the data that may signal unusual activity or inactivity on the part of the person wearing the independent activity detector 10.

At Step 115, the transmitted data is reviewed to ascertain whether unusual activity or inactivity has occurred. The determination of the manifestation of unusual activity or inactivity may be accomplished at the communication panel 30 or at an off-site monitoring center 40. If a determination is made that unusual activity or inactivity has occurred, then at Step 120, a caregiver is alerted.

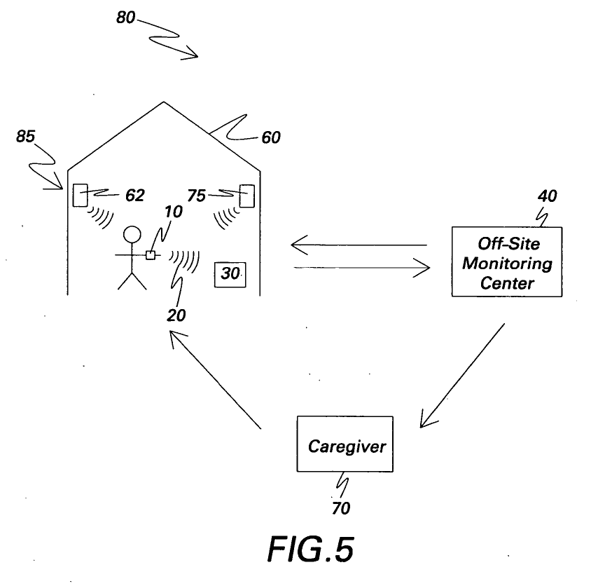

Next, with reference to FIG. 5 will be described an in-home monitoring system 80, which includes the independent activity detector 10, a house activity system 85, and the off-site monitoring center 40. The house activity system includes activity sensors 62, which may include motion sensors, exterior door sensors, and pressure sensors, such as, for example, pressure pads. A communication panel 30 is located in the house 60. Data from the activity sensors 62 and the independent activity detector 10 are transmitted to the communication panel 30, and are further transmitted to the off-site monitoring center 40. If the data indicates unusual activity or inactivity, a caregiver 70 is alerted. The caregiver 70 configures the sensitivity of the monitoring center 40. Thus, the caregiver 70 may provide instructions for the monitoring center 40 to contact the resident directly under certain circumstances (caretaker 70 unreachable, for example). The house activity system 85 also includes at least one physiological parameter sensors 75 configured to monitor one or more physiological parameters, such as, for example, weight or respiration.

Both the house activity system 85 and the independent activity detector system 50 may incorporate a temporal expectation function, as described fully in U.S. patent application Ser. No. 10/772,761, filed Feb. 4, 2004, the disclosure of which is incorporated herein by reference in its entirety.

The activity sensors 62 are positioned in various locations throughout a house 60, so positioned to be able to monitor and report on activity occurring in the house 60. For example, motion sensors are located in areas of the house 60 in which motion is likely to be detected, such as, for example, stairways, hallways, the kitchen, bathrooms, the living or family room, and bedrooms. Further, pressure pads may be located on articles of furniture upon which a resident of the house 60 is likely to spend time, such as, for example, the resident's bed or a favorite chair or at various locations throughout the house 60, such as incorporated within a bath mat or an area rug, for example. The combination of the motion sensors and the pressure pads allows one monitoring the data to ascertain a specific location of the resident within the house 60. For example, if a pressure pad on a living room chair indicates that a person is located on the chair, it can be assumed that the resident is on the living room chair. Contrarily, if there is no motion detected, no exterior door sensor has been triggered indicating the opening of the door, and no pressure pad has been triggered, the data would indicate a level of inactivity within the house 60. Depending upon the time of day and the duration, such inactivity may be considered unusual.

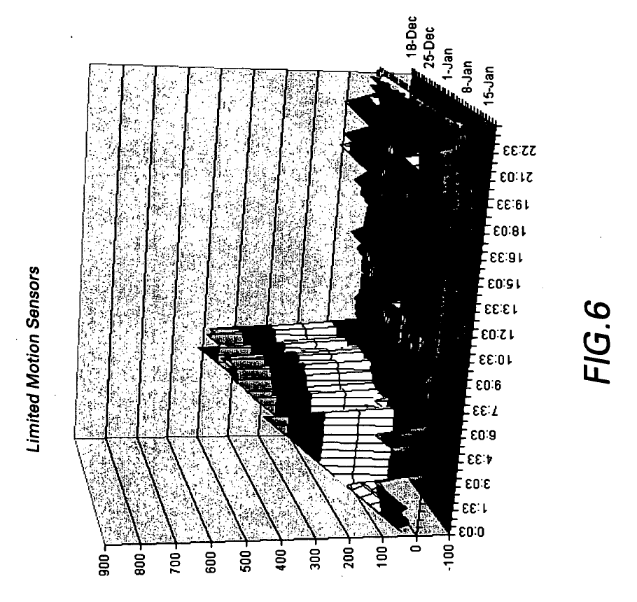

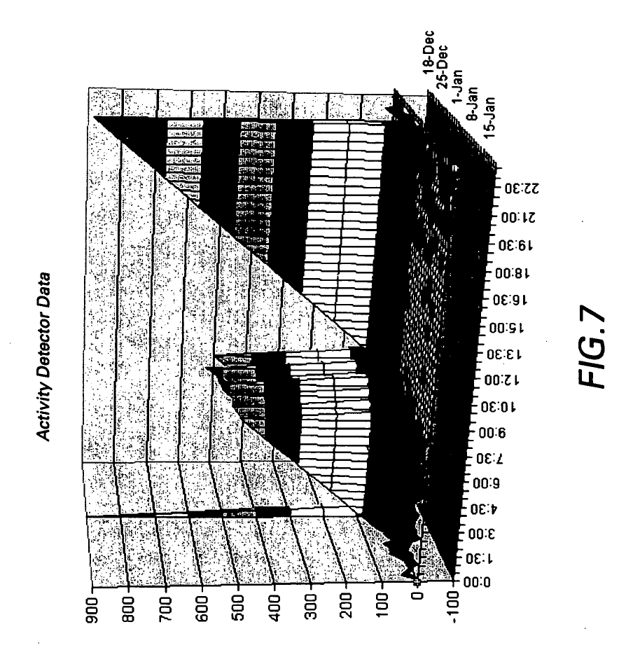

Data from the house activity system 85 at times may lack sufficient specificity to provide meaningful guidance as to the appropriateness of any given activity. The incorporation of the independent activity detector 10 within the in-home monitoring system 80 assists by adding greater specificity of individual movement within the house 60. For example, and with specific reference to FIGS. 6 and 7, there is shown a compilation of house activity detected with the house activity system 85 (FIG. 6) and individual activity detected with the independent activity detector 10 (FIG. 7). The higher and broader the peaks, the greater the amount of inactivity detected. Large blocks of inactivity are detected during the night, which would be expected. Other, smaller blocks of inactivity are detected throughout the day. One very large block of inactivity is noted on FIG. 7 throughout the day on December 25. Such a large block of inactivity may be caused by failure to wear the independent activity detector 10. In terms of greater specificity, there is a block of inactivity shown in FIG. 6 at around midday on January 15, indicating a certain level of inactivity within the house 60. However, referring to FIG. 7, there is no corresponding inactivity as detected by the independent activity detector 10, which indicates that the resident of the house 60 was wearing the independent activity detector 10 in the house 60 and was active in a location where the activity sensors 62 could not detect his activity. Thus, the independent activity detector 10 enables a more specific view of the actual activity ongoing within the house 60.

Next will be described another example of the greater specificity that data from the independent activity detector 10 lends to the analysis of activity within a house. As described previously, data packets 20 transmitted from the independent activity detector 10 to the communication panel include an authentication code or signal to identify the specific device sending the data, thereby allowing correlation of the data to a specific resident of the house 60. In a house 60 that includes more than one resident, the data from the independent activity detectors 10 can be used in conjunction with the data from the house activity system 85 to correlate specific activity, or inactivity, within the house with a specific resident. Or, if in a house 60 in which only one resident resides, and that resident is ill and consequently not very active, the inactivity of that resident can be monitored even in the presence of guests in the house that may trigger the motion sensors.

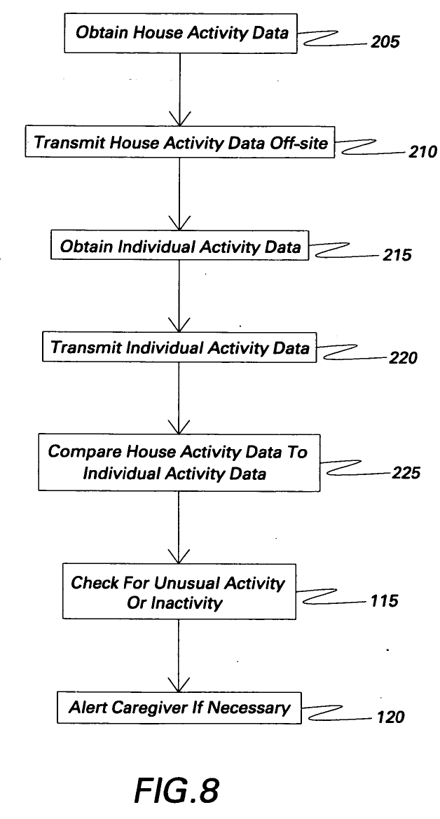

Referring now to FIG. 8, next will be described a method for determining, with an independent activity detector 10 and a house activity system 85, whether activity, or inactivity, of a resident of a house is unusual. At Step 205, house activity data is obtained. As described previously, the house activity data is taken from activity sensors 62, which wirelessly transmit the data to the communication panel 30. At Step 205, the house activity data is transmitted from the communication panel to the off-site monitoring center 40.

At Step 215, individual activity data is obtained. The individual activity data is obtained with the activity detection module 12 and is stored in the storage module 14. The individual activity data is then transmitted to the communication panel 30. The individual activity data may be analyzed at the communication panel 30 or may be analyzed off-site. At Step 220, the individual activity data is transmitted from the communication panel 30 to the off-site monitoring center 40.

At Step 225, the house activity data is compared to the individual activity data. Through such a comparison, discrepancies between the data can be determined and resolved. At Step 115, unusual activity or inactivity is checked, based upon the house activity data and the individual activity data. If unusual activity or inactivity is ascertained, the caregiver 70 is alerted at Step 120.

The combination of the independent activity detector 10 with the house activity system 85 provides more accurate, and greater resolution of, detection of falls and unusual activity/inactivity. This is especially true if the house activity system 85 has limited coverage due to the number or spacing of the activity sensors 62. This more accurate data may be used to trend patterns of activity for a period of time to help quantify changes in a risk of fall or other medical conditions over time. Additionally, this high quality data may allow for more precise monitoring of sleep patterns by providing greater ability to distinguish between sleep and a fall or other problem. High quality sleep measurements may be used as a measurement of overall wellness. The use of the independent activity detector 10 provides authentication of activity, thereby eliminating confusion between the activity levels of two or more residents within a single house 60 or between a resident and guests. Further, the independent activity detector 10 makes easier a determination as to whether a resident is at home or away. If the independent activity detector 10 fails to transmit data to the communication panel 30, it can be presumed that the resident has gone out of the house 60. The independent activity detector 10 will continue to store individual activity data for a length of time, such as, for example, eight or more hours, and that data can be communicated to the communication panel 30, and ultimately to the off-site monitoring center 40 upon return to the house 60. Further, the combination of the house activity system 85 with the independent activity detector 10 provides a location of the house 60 in which activity is taking place. For example, if the independent activity detector 10 indicates normal activity levels, but the house activity system 85 indicates that the activity has been taking place all day in the basement, such activity may be considered unusual.

In addition to incorporating results from the independent activity detector 10 and the house activity system 85 to ascertain the activities of residents, the results of other monitoring devices may be fused with the aforementioned results to ascertain the status of one or more physiological conditions of the resident. Specifically, physiological data, obtained with an appropriate physiological sensor 75, may be incorporated with data from the independent activity detector 10 and/or the house activity system 85. For example, respiratory data, obtained with a respiratory sensor, may be transmitted via another transmitter to the communication relay (communication panel 30) and then combined with data pertaining to a resident's activity level, SPO2, blood pressure and heart rate to remotely diagnose or monitor a physical condition, such as sleep apnea. Alternatively, correlation of a resident's diaphragm motion with a diaphragm monitor and heart rate with a heart rate monitor may be used to detect pain in the resident. Further correlation of data from the independent activity detector 10 and heart rate and sound may be used to detect a fall. As another example, data pertaining to a resident's weight as determined by a weight sensor may be incorporated with data from the independent activity detector 10 and/or the house activity system 85. Weight fluctuation, along with a decrease in activity level may signal the onset of one or more medical conditions requiring medical attention, such as, for example, congestive heart failure. Other physiological data to be incorporated with activity data may include ECG and temperature.

Another type of physiological data for incorporation is related to a resident's reaction to medication. For example, the titration of a drug dose may be incorporated with activity data. Fatigue is sometimes a side effect of a new drug, and by measuring the amount of the drug taken and correlating that with the activity level of the resident during the relevant period, appropriate dose levels of the new drug may be determined for the resident. More generally, certain combinations of drugs cause particularized reactions, and some resident's may have allergic reactions or other side effects to certain medications or combinations of medications. By incorporating a pharmacy/medicine knowledge base that contains medicine to medicine reactions and allergic reactions and other side effects to medicines, such as, for example, Firstdatabank.com, into activity data, a more complete analysis of how the resident may be feeling due to the medication(s) may be obtained.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described; but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. An independent activity detector configured to be associated with a person, comprising:
an activity detection module configured to collect data on activity of the person;
a storage mechanism configured to store the data collected by the activity detection module; and
a transmitter configured to transmit in at least quasi-real time the data collected by the activity detection module, wherein said storage mechanism is configured to retain a packet of data transmitted by said transmitter for retransmission by said transmitter.

2. The independent activity detector of claim 1, wherein said activity detection module is configured to ascertain whether the person is motionless.

3. The independent activity detector of claim 1, wherein said activity detection module comprises an accelerometer.

4. The independent activity detector of claim 1, wherein said activity detection module comprises a mercury switch.

5. The independent activity detector of claim 1, further comprising a mechanism for associating the independent activity detector with the person.

6. The independent activity detector of claim 1, wherein the independent activity detector is incorporated within a timepiece.

7. An independent activity detector configured to be associated with a person, comprising:
an activity detection module configured to collect data on activity of the person;
a storage mechanism configured to store the data collected by the activity detection module; and
a transmitter configured to transmit unfiltered, unanalyzed and in quasi-real time the data collected by the activity detection module, wherein said storage mechanism is configured to retain a packet of data transmitted by said transmitter for retransmission by said transmitter.

8. The independent activity detector of claim 7, wherein said activity detection module is configured to ascertain whether the person is immobile.

9. The independent activity detector of claim 7, wherein the independent activity detector is incorporated within a timepiece.

10. The independent activity detector of claim 7, wherein said activity detection module comprises at least one from the group consisting of a mercury switch and an accelerometer.

11. The independent activity detector of claim 7, further comprising a mechanism for associating the independent activity detector with the person.

12. The independent activity detector of claim 7, wherein a repeatable time period for the quasi-real time transmission of the data is no greater than every two hours.

13. The independent activity detector of claim 12, wherein a repeatable time period for the quasi-real time transmission of the data is no greater than fifteen minutes.

14. An independent activity detector system for use within a habitable structure, comprising:
an independent activity detector configured to be associated with a person, comprising:
an activity detection module configured to collect data on activity of the person;
a storage mechanism configured to store the data collected by the activity detection module; and
a transmitter configured to transmit in at least quasi-real time the data collected by the activity detection module, wherein said storage mechanism is configured to retain a packet of data transmitted by said transmitter for retransmission by said transmitter; and
a communication relay configured to receive the data transmitted by the transmitter.

15. The independent activity detector system of claim 14, wherein said activity detection module is configured to ascertain whether the person is motionless.

16. The independent activity detector system of claim 15, wherein said activity detection module comprises an accelerometer.

17. The independent activity detector system of claim 15, wherein said activity detection module comprises a mercury switch.

18. The independent activity detector system of claim 14, further comprising a mechanism for associating the independent activity detector with the person.

19. The independent activity detector system of claim 14, wherein the independent activity detector is incorporated within a timepiece.

20. The independent activity detector system of claim 14, wherein said communication relay is configured to analyze the data on activity of the person.

21. The independent activity detector system of claim 14, further comprising a monitoring center at a location separate from the habitable setting.

22. The independent activity detector system of claim 21, wherein said monitoring center is configured to analyze the data on activity of the person.

23. The independent activity detector system of claim 14, wherein said communication relay is configured to enhance efficiency of transmissions from said transmitter.

24. The independent activity detector system of claim 14, further comprising a physiological parameter sensor for sensing a physiological parameter of the person.

25. The independent activity detector system of claim 24, wherein the physiological parameter being sensed is one or more from the group consisting of respiration, weight, temperature, diaphragm motion, blood oxygenation, blood pressure, ECG, and drug dose amount.

26. The independent activity detector system of claim 24, wherein said physiological parameter sensor comprises a respiration sensor.

27. The independent activity detector system of claim 24, wherein said physiological parameter sensor comprises a weight sensor.

28. The independent activity detector system of claim 24, further comprising a pharmacy/medicine knowledge base.

29. An independent activity detector system for use within a habitable structure, comprising:
an independent activity detector configured to be associated with a person, comprising:
an activity detection module configured to collect data on activity of the person and configured to ascertain whether the person is motionless;
a storage mechanism configured to store the data collected by the activity detection module; and
a transmitter configured to transmit unfiltered, unanalyzed and in quasi-real time the data collected by the activity detection module, wherein said storage mechanism is configured to retain a packet of data transmitted by said transmitter for retransmission by said transmitter; and
a communication relay configured to receive the data transmitted by the transmitter.

30. The independent activity detector system of claim 29, wherein the independent activity detector is incorporated within a timepiece.

31. The independent activity detector system of claim 29, wherein said communication relay is configured to analyze the data on activity of the person.

32. The independent activity detector system of claim 29, further comprising a monitoring center at a location separate from the habitable setting.

33. The independent activity detector system of claim 29, wherein said monitoring center is configured to analyze the data on activity of the person.

34. The independent activity detector system of claim 29, wherein said activity detection module comprises at least one from the group consisting of a mercury switch and an accelerometer.

35. An independent activity detector system for monitoring a person's health, comprising:
an independent activity detector configured to be associated with a person, comprising:
an activity detection module configured to collect activity data on activity of the person;
a storage mechanism configured to store the activity data collected by the activity detection module; and
a first transmitter configured to transmit unfiltered, unanalyzed and in at least quasi-real time the activity data collected by the activity detection module;
a physiological sensor for obtaining physiological data pertaining to the person;
a second transmitter configured to transmit physiological data collected by the activity detection module; and
a communication relay configured to receive the activity and physiological data transmitted by the first and second transmitters, wherein the physiological data and the activity data are correlated to determine a status of at least one physiological condition of the person.

36. The independent activity detector system of claim 34, wherein the physiological parameter being sensed is one or more from the group consisting of respiration, weight, temperature, diaphragm motion, blood oxygenation, blood pressure, ECG, and drug dose amount.

37. The independent activity detector system of claim 35, wherein the physiological sensor comprises a respiratory sensor.

38. The independent activity detector system of claim 35, wherein the physiological sensor comprises a weight sensor.

39. The independent activity detector system of claim 35, wherein the physiological sensor comprises a heart rate monitor and a diaphragm motion monitor.

40. The independent activity detector system of claim 35, further comprising a pharmacy/medicine knowledge base.

41. A method for detecting unusual activity or inactivity of a person within a habitable structure, comprising:
detecting individual activity data of the person with an independent activity detector;
transmitting the individual activity data in at least quasi-real time to an analyzing mechanism; and
ascertaining whether activity or inactivity of the person detected with the independent activity detector is unusual.

42. The method of claim 41, further comprising alerting a caregiver if said ascertaining step leads to a conclusion that the activity or inactivity of the person obtained with the independent activity detector is unusual.

43. The method of claim 41, wherein said transmitting comprises transmitting the individual activity data in quasi-real time to a communication relay.

44. The method of claim 43, wherein said communication relay functions as the analyzing mechanism.

45. The method of claim 43, wherein said transmitting further comprises transmitting the individual activity data in quasi-real time from the communication relay to a monitoring center at a separate location from the habitable structure.

46. The method of claim 43, wherein the monitoring center functions as the analyzing mechanism.

47. The method of claim 41, wherein said transmitting comprises transmitting a signal for authenticating the individual activity data to a specific person.

48. A method for detecting unusual activity or inactivity of a person within a habitable structure, comprising:
detecting activity data in the habitable structure;
detecting individual activity data of the person;
transmitting the individual activity data in quasi-real time to an analyzing mechanism;
comparing the activity data of the habitable structure to the individual activity data; and
checking for unusual activity or unusual inactivity of the person.

49. The method of claim 48, further comprising transmitting the activity data of the habitable structure to the analyzing mechanism.

50. The method of claim 49, wherein said transmitting the activity data of the habitable structure is accomplished in quasi-real time.

51. The method of claim 49, wherein said transmitting the individual activity data comprises transmitting a signal for authenticating the individual activity data to a specific person.

52. The method of claim 48, further comprising alerting a caregiver if said checking step leads to a conclusion that the activity or inactivity of the person is unusual.

53. The method of claim 48, wherein said detecting activity data of the habitable structure comprises obtaining the activity data with the use of one or more activity sensors positioned throughout the habitable structure.

54. The method of claim 48, wherein said checking comprises ascertaining whether the person is motionless.
